(19) United States
(12) Patent Application Publication
(10) Pub. No.: US 2013/0122577 A1
(43) Pub. Date: May 16, 2013

(54) KIT INCLUDING SEQUENCE SPECIFIC BINDING PROTEIN AND METHOD AND DEVICE FOR DETERMINING NUCLEOTIDE SEQUENCE OF TARGET NUCLEIC ACID

(71) Applicants: Joo-won Rhee, Yongin-si (KR); Su-hyeon Kim, Seoul (KR); Jeong-gun Lee, Seoul (KR); Mi-jeong Song, Suwon-si (KR)

(72) Inventors: Joo-won Rhee, Yongin-si (KR); Su-hyeon Kim, Seoul (KR); Jeong-gun Lee, Seoul (KR); Mi-jeong Song, Suwon-si (KR)

(73) Assignee: Samsung Electronics Co., Ltd., Suwon-si, Gyeonggi-do (KR)

(21) Appl. No.: 13/577,418

(22) PCT Filed: Feb. 7, 2011
(86) PCT No.: PCT/KR2011/000778

§ 371 Date: Jan. 23, 2013

(30) Foreign Application Priority Data
Feb. 4, 2010 (KR) 10-2010-0010500

(51) Int. Cl. C12Q 1/68

(52) U.S. Cl. CPC C12Q 1/6869; Y10S 977/774; Y10S 977/92; B82Y 15/00
USPC 435/287.2; 436/501; 530/350; 536/23.4; 422/69; 977/774; 977/920

(57) ABSTRACT

Provided are kits for determining a nucleotide sequence of a target nucleic acid, the kit including at least one sequence specific binding protein and a detectable tag. In accordance with a kit for determining a nucleotide sequence of a target nucleic acid according to one exemplary embodiment and a method and device for determining a nucleotide sequence of a target nucleic acid, the nucleotide sequence of the target nucleic acid may be more efficiently determined.

### TECHNICAL FIELD

The present disclosure relates to a kit including a sequence specific binding protein, and a method and device for determining a nucleotide sequence of a target nucleic acid.

### BACKGROUND ART

The analysis of nucleotide sequences is an elementary tool in medical and biological research. It may be used as means for discovery of drug targets or diagnostic markers, observation of mutations in an individual genome, and securing useful biological resources, and may also be used even in the diagnostic area of diseases caused by genetic mutations, for example, disorders such as hereditary diseases, cancers, etc.

Examples of nucleotide analysis methods include two basic approaches such as the chain termination method and the chemical degradation method. Both methods require that a DNA fragment, which may be differentiated from a larger DNA fragment by single nucleotides, needs to be separated by high-resolution gel electrophoresis. Since these processes limit the size of DNA, which may be determined at one time, a lot of costs and time are needed and it is difficult to analyze many specific sequences at one time.

Since then, the emergence of next-generation techniques of gene sequence analysis taking the place of the chain termination method and the chemical degradation method has improved the efficiency of gene analysis and also greatly reduced the costs of analysis. Although many samples may be simultaneously analyzed by the next-generation techniques of gene sequence analysis, it takes a lot of time and the read-length of the sequence is so short (about 25 bp to about 500 bp) that many multiple sequences need to be analyzed. Also, it is difficult to detect structural mutations of DNA or to analyze the copy number of genes.

The related art discloses a method for analyzing a sequence of DNA by immobilizing a DNA molecule of about 100 kb extracted from a living organism on a nano-channel and subjecting it to nicking using an endonuclease and a site specific probing technique. However, it is difficult to differentiate the sequence of a target nucleotide with high-resolution.

Therefore, there is still a need to develop methods and devices for determining a nucleotide sequence of a target nucleic acid more efficiently, even though they are based on the related art.

### DISCLOSURE OF INVENTION

### Technical Problem

Provided are kits for determining a nucleotide sequence of a target nucleic acid, the kit including at least one sequence specific binding protein and a detectable tag.

Provided are methods and devices for determining a nucleotide sequence of a target nucleic acid.

Additional aspects will be set forth in part in the description which follows and, in part, will be apparent from the description, or may be learned by practice of the presented embodiments.

### Solution to Problem

According to an aspect of the present invention, a kit for determining a nucleotide sequence of a target nucleic acid, the kit includes at least one sequence specific binding protein and a detectable tag.

As used herein, the term “nucleic acid” refers to a polymer of nucleotides. The nucleic acid may include deoxyribonucleic acid (DNA; gDNA and cDNA) and/or ribonucleic acid (RNA), peptide nucleic acid (PNA), or locked nucleic acid (LNA). Nucleotides, which are the basic building blocks of nucleic acids, include not only natural nucleotides such as deoxyribonucleotide and ribonucleotide, but also artificial analogues including a modified sugar or base.

As used herein, the term “target nucleic acid” refers to a nucleic acid whose nucleotide sequence is to be determined. The target nucleic acid may include genomic DNA, mRNA, cDNA, or DNA amplified by amplification reaction, but is not limited thereto.

As used herein, the term “sequence specific binding protein” refers to a kind of protein capable of specifically detecting and binding to a nucleotide sequence of a target nucleic acid. The term “motif” refers to a particular amino acid sequence specifically recognizes a particular nucleotide sequence of a target nucleic acid. The motif may include a tertiary structure and/or secondary structure as well as a primary amino acid sequence. The motif may specifically recognize a single stranded or a double stranded nucleic acid. The sequence specific binding protein may include at least one motif. In some embodiments, the motif may be selected from the group consisting of a zinc finger motif, a helix-turn-helix motif, a helix-loop-helix motif, a leucine zipper motif, a nucleic acid-binding motif of restriction endonuclease, and a combination thereof.

In some embodiments, the sequence specific binding protein may include one to five zinc finger motifs, and in some embodiments, may include one to three zinc finger motifs. Most preferably, the sequence specific binding protein may include two zinc finger motifs.

The zinc finger motif may have any of various backbone structures, and in some embodiments, may be selected from the group consisting of Cys₂His₂, Cys₄, His₄, His₃Cys, Cys₃X, His₃X, Cys₂X₂, His₂X₂ (wherein X is a zinc ligating amino acid) and combinations thereof, which are nonlimiting examples of backbone structures. The zinc finger motif may have a particular amino acid sequence containing conserved cysteine and histidine residues, for example, CXX(XX)CXXXXXXXXXXXXHXXXH. The zinc finger motif is found in a widely varying family of DNA-binding proteins. The conserved cysteine and histidine residues in this motif form ligands to a zinc ion whose coordination is essential to stabilize the tertiary structure. Conservation is sometimes of a class of residues rather than a specific residue: for example, in the 12-residue loop between the zinc ligands, one position is preferentially hydrophobic, specifically leucine or phenylalanine.

The zinc finger motif may specifically recognize and bind to a target nucleotide sequence. The target nucleotide sequence may have a nucleotide length of about 3 to about 15, and in some embodiments, may have a nucleotide length of about 6 to about 12. For example, a Cys2His2 zinc finger motif may include a-helical seven amino acids that specifically recognize three nucleotide sequences. Zinc finger motifs may specifically recognize different nucleotide sequences. Nucleotide sequences specifically recognized by certain amino acid sequences of zinc finger motifs are disclosed in http://www.scripps.edu/mb/barbas/zfdesign/zfdesignhome.php.

The zinc finger motif may be a wild type, a mutant type, or a combination thereof. A mutant zinc finger motif may include about 1 to about 5 amino acid residues substituting those of a wild type zinc finger motif, and in some embodiments, may include about 2 to about 4 of such amino acid residues. These amino acid residue substituents may specifically bind to a nucleic acid.

A library of zinc finger motifs capable of specifically recognizing and binding to specific nucleotide sequences may be constructed by random mutation on the gene level. For example, a phage display method by which a zinc finger motif library is displayed on a phage surface, a yeast one-hybrid method, a bacterial two-hybrid method, or a cell-free translation may be used to screen zinc finger motifs.

In some embodiments, the sequence specific binding protein may be linked with a detectable tag.

In some embodiments, the kit may include at least two or more sequence specific binding proteins, wherein at least two or more of the sequence specific binding proteins have different detectable tag. The kit may include two or more different sequence specific binding protein and detectable tags. Each of the sequence specific binding proteins may have different detectable tag. For example, the first sequence specific binding protein contained in the kit may be labeled with GFP, the second sequence specific binding protein contained in the kit may be labeled with YFP, the third sequence specific binding protein contained in the kit may be labeled with RFP, and so on. Each of different sequence specific binding protein and detectable tag may bind to different specific sequence in the target nucleic acid and be used to determine the nucleotide sequence of the target nucleic acid. By containing two or more different sequence specific binding protein and detectable tags in the kit, the kit can be used to determine a nucleotide sequence in a target nucleic acid with increased accuracy compared to a kit containing only one kind of the sequence specific binding protein and the detectable tag.

As used herein, the term “detectable tag” refers to an atom or a molecule used to specifically detect a molecule or substance including a label, from among the same type of molecules or substances without a label. The detectable tag may include at least one selected from the group consisting of a tag emitting a light signal, a tag emitting electrical signal, a tag emitting a radioactivity and a combination thereof. The tag emitting a light signal may include a fluorescent material and phosphorescent material as well as a material having a specific pattern of light absorbing and/or emitting. The fluorescent material may include a fluorescent protein which emits light upon exposed to a light. The fluorescent protein may include a fluorescent protein selected from the group consisting of a yellow fluorescent protein (YFP), a green fluorescent protein (GFP), a red fluorescent protein (RFP) and a combination thereof. The GFP is a protein composed of 23 amino acid residues (26.9 KDa) that exhibits bright green fluorescence when exposed to blue light. Although many other marine organisms have similar green fluorescent proteins, GFP traditionally refers to the protein first isolated from the jellyfish Aequorea Victoria. The GFP from A. Victoria has a major excitation peak at a wavelength of 395 nm and a minor one at 475 nm. Its emission peak is at 509 nm, which is in the lower green portion of the visible spectrum. The GFP from the sea pansy (Renilla reniformis) has a single major excitation peak at 498 nm. The GFP used herein includes GFP derivative as well as a wild type GFP. Due to the potential for widespread usage and the evolving needs of researchers, many different mutants of GFP have been engineered. The GFP derivative may include GFP having a single point mutation (S65T) reported in 1995 in Nature by Roger Tsien. This mutation dramatically improved the spectral characteristics of GFP, resulting in increased fluorescence, photostability, and a shift of the major excitation peak to 488 nm, with the peak emission kept at 509 nm. A 37° C. folding efficiency (F64L) point mutant to this S65T mutant yielding enhanced GFP (EGFP) was discovered in 1995 by the lab of Ole Thastrup. EGFP allowed the practical use of GFPs in mammalian. Many other mutations have been made, including color mutants; in particular, blue fluorescent protein (EBFP, EBFP2, Azurite, mKalama1), cyan fluorescent protein (ECFP, Cerulean, CyPet), and yellow fluorescent protein derivatives (YFP, Citrine, Venus, YPet). BFP derivatives (except mKalama1) contain the Y66H substitution. The critical mutation in cyan derivatives is the Y66W substitution, which causes the chromophore to form with an indole rather than phenol component. Several additional compensatory mutations in the surrounding barrel are required to restore brightness to this modified chromophore due to the increased bulk of the indole group. The red-shifted wavelength of the YFP derivatives is accomplished by the T203Y mutation and is due to π-electron stacking interactions between the substituted tyrosine residue and the chromophore. These two classes of spectral variants are often employed for fluorescence resonance energy transfer (FRET) experiments. Genetically-encoded FRET reporters sensitive to cell signaling molecules, such as calcium or glutamate, protein phosphorylation state, protein complementation, receptor dimerization, and other processes provide highly specific optical readouts of cell activity in real time. The YFP is a genetic mutant of GFP, derived from Aequorea Victoria. Its excitation peak is 514 nm and its emission peak is 527 nm. Like green fluorescent protein (GFP), it is a useful tool in cell and molecular biology, usually explored using fluorescence microscopy. Three improved versions of YFP are Citrine, Venus, and Ypet. They have reduced chloride sensitivity, faster maturation, and increased brightness (product of the extinction coefficient and quantum yield). Typically, yellow FPs serve as the acceptor for genetically-encoded FRET sensors of which the most likely donor FP is mCFP (monomeric cyan FP). The red-shift relative to GFP is caused by a Pi-Pi stacking interaction as a result of the T203Y mutation, which essentially increases the polarizability of the local chromophore environment as well as providing additional electron density into the chromophore.

The RFP is a red-emitting fluorescent protein. The first coral-derived fluorescent protein to be extensively utilized was derived from Discosoma striata and is commonly referred to as DsRed. Once fully matured, the fluorescence emission spectrum of DsRed features a peak at 583 nm whereas the excitation spectrum has a major peak at 558 nm and a minor peak around 500 nm. DsRed is an obligate tetramer and can form large protein aggregates in living cells. The RFP used herein includes derivatives of wild type DsRed. A few of the problems with DsRed fluorescent proteins have been overcome through mutagenesis. The second-generation DsRed, known as DsRed2, contains several mutations at the peptide amino terminus that prevent formation of protein aggregates and reduce toxicity. In addition, the fluorophore maturation time is reduced with these modifications. The DsRed2 protein still forms a tetramer, but it is more compatible with green fluorescent proteins in multiple labeling experiments due to the quicker maturation. Further reductions in maturation time have been realized with the third generation of DsRed mutants, which also display an increased brightness level in terms of peak cellular fluorescence. Red fluorescence emission from DsRed-Express can be observed within an hour after expression, as compared to approximately six hours for DsRed2 and 11 hours for DsRed. A yeast-optimized variant, termed RedStar, has been developed that also has an improved maturation rate and increased brightness. The presence of a green state in DsRed-Express and RedStar is not apparent, rendering these fluorescent proteins the best choice in the orange-red spectral region for multiple labeling experiments. Because these probes remain obligate tetramers, they are not the best choice for labeling proteins. Several additional red fluorescent proteins showing a considerable amount of promise have been isolated from the reef coral organisms. One of the first to be adapted for mammalian applications is HcRed1, which was isolated from Heteractis crispa and is now commercially available. HcRed1 was originally derived from a non-fluorescent chromoprotein that absorbs red light through mutagenesis to produce a weakly fluorescent obligate dimer having an absorption maximum at 588 nm and an emission maximum of 618 nm. Although the fluorescence emission spectrum of this protein is adequate for separation from DsRed, it tends to co-aggregate with DsRed and is far less bright.

The detectable tag may further include a colored bead, an antigen determinant, an enzyme, hybridizable nucleic acid, a chromophore, an electrically detectable molecule, a molecule providing modified fluorescence-polarization or modified light-diffusion, a quantum dot, or the like. In addition, the detectable tag may be radioactive isotopes such as P³² and S³⁵, a chemiluminescent compound, labeled binding protein, a heavy metal atom, a spectroscopic marker such as a dye, or a magnetic label. The dye may include quinoline dye, triarylmethane dye, phthalene, azo dye, or cyanine dye, but is not limited thereto. Nonlimiting suitable fluorescent materials may include Alexa Fluor 350, Alexa Fluor 430, Alexa Fluor 488, Alexa Fluor 532, Alexa Fluor 546, Alexa Fluor 568, Alexa Fluor 594, Alexa Fluor 633, Alexa Fluor 647, Alexa Fluor 660, Alexa Fluor 680, Cy2, Cy3.18, Cy3.5, Cy3, Cy5.18, Cy5.5, Cy5, Cy7, Oregon Green, Oregon Green 488-X, Oregon Green, Oregon Green 488, Oregon Green 500, Oregon Green 514, SYTO 11, SYTO 12, SYTO 13, SYTO 14, SYTO 15, SYTO 16, SYTO 17, SYTO 18, SYTO 20, SYTO 21, SYTO 22, SYTO 23, SYTO 24, SYTO 25, SYTO 40, SYTO 41, SYTO 42, SYTO 43, SYTO 44, SYTO 45, SYTO 59, SYTO 60, SYTO 61, SYTO 62, SYTO 63, SYTO 64, SYTO 80, SYTO 81, SYTO 82, SYTO 83, SYTO 84, SYTO 85, SYTOX Blue, SYTOX Green, SYTOX Orange, SYBR Green YO-PRO-1, YO-PRO-3, YOYO-1, YOYO-3, and thiazole orange, but is not limited thereto.

In some embodiments, the sequence specific binding protein and the detectable tag may be coupled by a linker, which specifically binds to the sequence specific binding protein and the detectable tag. The linker may be attached to, for example, the N-terminus or C-terminus of the sequence specific binding protein. The linker may be a non-peptide linker or a peptide linker.

The non-peptide linker may be any of various compounds that may be used as linkers in the art. A suitable linker may be selected based on the type of a functional group in a protein or peptide. For example, the linker may be an alkyl linker or an amino linker. The alkyl linker may be a branched or non-branched, cyclic or acylic, substituted or unsubstituted, saturated or unsaturated, chiral, achiral or racemic mixture. For example, the alkyl linker may have about 2 to about 18 carbon atoms. Other suitable alkyl linkers may include at least one functional group selected from among, but not limited to, hydroxy, amino, thiol, thioether, ether, amide, thioamide, ester, urea, and thioether. The alkyl linker may include a 1-propanol linker, a 1,2-propandiol linker, a 1,2,3-propantriol linker, a 1,3-propandiol linker, a triethylene glycol hexaethylene glycol linker, a polyethylene glycol linker (for example, [—O—CH₂—CH₂—]ₙ (n=1-9)), a methyl linker, an ethyl linker, a propyl linker, a butyl linker, or a hexyl linker.

The peptide linker may be any of various linkers that are widely used in the art, and for example, may be a linker including a plurality of amino acid residues. The peptide linker may allow the sequence specific binding protein and the detectable tag (for example, a fluorescent protein) to be spaced apart from each other by a distance that is sufficient enough for each polypeptide to fold in appropriate secondary and tertiary structures. For example, the peptide linker may include Gly, Asn and Ser residues, and in some other embodiments, may include neutral amino acid residues, such as Thr and Ala. Amino acid sequences suitable for the peptide linker are known in the art. Suitable amino acid sequences may include (Gly₄-Ser)₃, (Gly₂-Ser)₂, and Gly₄-Ser-Gly₅-Ser. The linker may be unnecessary, and may have various lengths, as long as it does not affect functions of the sequence specific binding protein and the detectable tag.

In some embodiments, the kit may further include a target nucleic acid. The target nucleic acid may be double-stranded, and may have a length of about 1 kb to about 10 Mb.

The kit may include a reagent for stabilizing the specific sequence-binding protein. For example, the kit may include a buffer solution known in the art. The kit may be manufactured to have a plurality of separate packages or compartments.

According to another aspect of the present invention, a gene construct comprises a polynucleotide encoding a fluorescent protein fused with a zinc finger protein, operatively linked to a promoter.

As used herein, the term “gene construct” refers to a functional unit necessary for the expression of a gene of interest. The gene construct may include a vector. The term “vector” refers to a vector used to express a target gene in a host cell. For example, the vector may include a plasmid vector, a cosmid vector, and a virus vector, such as a bacteriophage vector, an adenovirus vector, a retrovirus vector, and an adeno-associated virus vector. Suitable recombinant vectors may be constructed by manipulating plasmids that are widely used in the art, such as pSC101, pGV1106, pACYC177, Co1E1, pKT230, pME290, pBR322, pUC8/9, pUC6, pBD9, pHC79, pIJ61, pLAFR1, pHV14, pGEX series, pET series, and pUC19; phages, such as λgt4λB, λ-Charon, λαz1, and M13; or viruses, such as SV40.

The fluorescent protein and zinc finger protein may be as described above. The fluorescent protein may be one selected from the group consisting of a yellow fluorescent protein (YFP), a green fluorescent protein (GFP), a red fluorescent protein (RFP) and a combination thereof. The fluorescent protein and the zinc finger protein may be fused in order N terminus to C terminus or C terminus to N terminus. The fluorescent protein and the zinc finger protein may be fused by a linker for example, peptide or nonpeptide linker.

In the gene construct, the sequence of the polynucleotide coding for the fusion protein may be operatively linked to a promoter. As used herein, the term “operatively linked” indicates a functional linkage between a nucleic acid expression control sequence (e.g., a promoter sequence and/or a terminator sequence) and another nucleic acid sequence, wherein the nucleic acid expression control sequence may control transcription and/or translation of the other nucleic acid sequence thereby.

The gene construct may be an expression vector, that is a recombinant vector, that stably expresses the fusion protein in a host cell. The expression vector may be a conventional vector that is used in the art to express an exogenous protein in plants, animals, or microorganisms. The recombinant vector may be constructed using various methods known in the art.

The recombinant vector may be constructed using a prokaryotic cell or a eukaryotic cell as a host. For example, if the recombinant vector is an expression vector and a prokaryotic cell is used as a host cell, the vector may include a promoter capable of initiating transcription, such as pL^{λ} promoter, trp promoter, lac promoter, tac promoter, and T7 promoter, a ribosome-binding site to initiate translation, and a transcription/translation termination sequence. If a eukaryotic cell is used as a host cell, an origin of replication operating in the eukaryotic cell included in the vector may include a f1 replication origin, a SV40 replication origin, a pMB 1 replication origin, an adeno replication origin, an AAV replication origin, or a BBV replication origin, but is not limited thereto. The promoter used in the recombinant vector may be a promoter derived from a genome of a mammal cell (for example, a metallothionein promoter) or a promoter derived from a virus of a mammal cell (for example, an adenovirus anaphase promoter, a vaccinia virus 7.5K promoter, a SV40 promoter, a cytomegalovirus promoter, or a tk promoter of HSV) and may include a polyadenylated sequence as a transcription termination sequence.

Any host cell known in the art to enable stable and continuous cloning or expression of the recombinant vector may be used. Suitable prokaryotic host cells may include E. coli JM109, E. coli BL21, E. coli RR1, E. coli LE392, E. coli B, E. coli X 1776, E. coli W3110, Bacillus genus strains such as Bacillus subtillis or Bacillus thuringiensis, intestinal bacteria and strains such as Salmonella typhymurium, Serratia marcescens, and various Pseudomonas species. Suitable eukaryotic host cells to be transformed may include yeasts, such as Saccharomyce cerevisiae, insect cells, plant cells, and animal cells, for example, Chinese hamster ovary (CHO) cell lines, W138, BHK, COS-7, 293, HepG2, 3T3, RIN, and MDCK cell lines.

The polynucleotide or the recombinant vector including the polynucleotide may be transferred into a host cell by using known transfer methods. Suitable transfer methods may be chosen according to the host cell. Suitable transfer methods for prokaryotic host cells may include a method using CaCl₂ and electroporation. Suitable transfer methods for eukaryotic host cells may include microinjection, calcium phosphate precipitation, electroporation, liposome-mediated transfection, and gene bombardment. However, any suitable transfer method may be used.

The transformed host cell may be screened using a phenotype expressed by a selected marker, and known methods. For example, if the selected marker is a gene that is resistant to a specific antibiotic, a transformed host cell may be easily screened by being cultured in a medium containing the antibiotic.

According to another aspect of the present invention, a device for determining a nucleotide sequence of a target nucleic acid includes: a sample injection unit for injecting a target nucleic acid and a sequence specific binding protein and a detectable tag; a sample transportation unit comprising a channel fluidically connected to the sample injection unit; a fluid flow control unit for controlling a flow of the sample; a detecting unit for detecting a signal from the detectable tag.

The channel in the sample transportation unit have a dimension to allow a target nucleic acid to be passed therethrough or the sequence specific binding protein and the detectable tag to bind to the target nucleic acid. For example, the channel may have a depth and a width of about 30 nm to about 200 nm, respectively.

The device may further include a sample waste unit fluidically connected to the sample transportation unit and disposed in the opposite end of a channel to which the sample injection unit is connected. The channel may have the same dimension as that of the channel in the sample transportation unit.

In addition, the sample transportation unit may allow one end of each channel in at least two channels to be sequentially and fluidically connected to the other end. The device may further include a sample recycling unit fluidically connected to one end of the channel. The sample recycling unit may include a proteolytic enzyme. Also, the device further comprises a sample labeling unit fluidically connected to the sample recycling unit, disposed at the other end of the channel to which the sample recycling unit is connected. The sample labelling unit may include a sequence specific binding protein comprising at least one motif and a detectable tag. As used herein, the term “proteolytic enzyme” refers to any enzyme that conducts proteolysis, that is, begins protein catabolism by hydrolysis of the peptide bonds that link amino acids together in the polypeptide chain forming the protein. The proteolytic enzyme may be used to remove the sequence specific binding protein to recycle the target nucleic acid. The proteolytic enzyme includes, for example, peptidase, proteinase K, serine proteases, threonine proteases, cysteine proteases, aspartate proteases, metalloproteases or glutamic acid proteases, but is not limited thereto.

The device may further comprise an operation unit for converting a signal detected from the detectable tag into a nucleotide sequence which corresponds to the signal.

According to another aspect of the present invention, a method comprises: contacting a target nucleic acid with at least one sequence specific binding protein and a detectable tag; detecting a signal from the detectable tag and determining the nucleic acid sequence of the target nucleic acid sequence from the signal.

In some embodiments the contacting may be achieved by mixing the sequence specific binding protein and the target nucleic acid in a liquid medium. The liquid medium may be any buffer solution known in the art to maintain stabilities of the sequence specific binding protein and the target nucleic acid and to be able to bind the sequence specific binding protein and the target nucleic acid. The contacting allows a motif of the sequence specific binding protein to approach the target nucleic acid, and the protein to specifically bind to a nucleotide sequence of interest of the target nucleic acid. The contacting may be followed by washing out any sequence specific binding protein that remains unbound.

The target nucleic acid may be double-stranded. In addition, the target nucleic acid may be prepared having various lengths by using known methods in the art. For example, the target nucleic acid may have a length of about 1 kb to about 10 Mb, and in some embodiments, may have a length of about 10 kb to about 10 Mb.

The method may further include introducing the contacted sample into a channel. The channel may have a dimension to allow a target nucleic acid to be passed therethrough or the sequence specific binding protein and the detectable tag to bind to the target nucleic acid. For example, the channel may have a depth and a width of about 30 nm to about 200 nm, respectively. The sample may be introduced into the channel by any known method. For example, the sample may be introduced into the channel by a mechanical pumping, electrical driving force, or pressure drop. The sample may be used for detect the position where the sequence specific binding protein and the detectable tag bind to the target nucleic acid in a static or flowing conditions within the channel.

The method may include detecting a signal from the detectable tag. Examples of the detectable tag are the same as described above in conjunction with the kit. The detecting signal may be performed by any known method in the art. The signal may be detected by using any device that measures fluorescence, for example, a fluorometer or fluorescence microscopy when the tag is a fluorescent material or a fluorescent protein. The detecting may include detecting the position where the sequence specific binding protein and the detectable tag bind to the target nucleic acid. The position may a relative position from a certain position in the target nucleic acid, for example, each of the end positions of the target nucleic acid. The position may be detected after the sample is introduced into a channel. In this case, the position may be measured in conjunction with a predetermined position in the channel.

In the determining of the sequence, a detection signal may be identified by detecting the signal generated from the detectable tag by using a detector. In some embodiments, examples of signals generated from the detectable tag include a signal selected from the group consisting of a magnetic signal, an electric signal, a light emitting signal such as a fluorescent or Raman signal, a diffused light signal, and a radioactive signal. Examples of the detection signal are the same as described above in conjunction with the detectable tag.

In some embodiments, the determining of the sequence may be performed by determining a specific sequence from the detected signal and identifying a position where the signal is detected in the channel. For example, when a GFP and a YFP are used as a detectable tag, a green fluorescent light corresponding to GFP signal indicates that there is a particular nucleotide sequence where the GFP binds to, and a yellow fluorescent light corresponding to YFP signal indicates that there is a particular nucleotide sequence where the YFP binds to. In this way, the nucleotide composition of the target nucleic acid can be determined. The two or more different detectable tags, for example, fluorescent proteins may be used to determine the nucleotide composition of the target nucleic acid, if desired. Further, one nucleotide composition of the target nucleic acid obtained from the one detectable tag may be combined with another nucleotide composition of the target nucleic acid obtained from the another detectable tag, if desired, to increase an accuracy for the composition.

The position where the sequence specific binding protein and the detectable tag bind to may be identified by in static or flowing conditions within a channel. For example, the position where the sequence specific binding protein and the detectable tag bind to may be identified by in static conditions, that is, the position of the target nucleic acid remains unchanged in the channel. The target nucleic acid may be fixed to the channel at one end and the position where the sequence specific binding protein and the detectable tag bind to may be identified from the fixed point to the other end of the nucleic acid, or the position where the sequence specific binding protein and the detectable tag bind to may be identified from the fixed point of the channel. The position where the sequence specific binding protein and the detectable tag bind to may be identified by in flowing conditions. That is, the target nucleic acid which bound to the sequence specific binding protein and the detectable tag passes through the channel from one end to the other end, while the signal from the detectable tag is measured with a time interval. In this case, the position where the sequence specific binding protein and the detectable tag bind to may be identified from the one end to the other end with a time interval. The nucleotide sequence of the target nucleic acid may be determined by combining the obtained information about the position where the motif binds to and the sequence to which the motif binds.

In addition, the method may be performed by determining a partial or whole nucleotide sequence of the target nucleic acid.

By the method, the signal may be detected to identify a nucleotide sequence of a nucleic acid to which the corresponding sequence specific binding protein and the detectable tag bind. When the method for determining a nucleotide sequence for a target nucleic acid is continuously repeated by varying the kinds of the sequence specific binding proteins and detectable tags and the thus-obtained nucleotide sequences are combined, a whole nucleotide sequence of a target nucleic acid may be determined. In addition, the repetition may be performed by removing the sequence specific binding protein the detectable tag using a proteolytic enzyme such as protease followed by recycling the target nucleic acid.

One or more embodiments of the present invention will be described in further detail with reference to the following examples. These examples are for illustrative purposes only and are not intended to limit the scope of the one or more embodiments of the present invention.

### BRIEF DESCRIPTION OF DRAWINGS

These and/or other aspects will become apparent and more readily appreciated from the following description of the embodiments, taken in conjunction with the accompanying drawings of which:

FIG. 1 is a schematic view of an device for determining a nucleotide sequence, including a channel in a sample transportation unit according to one exemplary embodiment;

FIG. 2 is a schematic view of an device for determining a nucleotide sequence, including at least two channels in a sample transportation unit according to one exemplary embodiment;

FIG. 3 is a map of a vector pEP21b-YFP-ZF for expressing a sequence specific binding protein with which a fluorescent protein (YFP) according to one exemplary embodiment is linked;

FIG. 4 is a result of non-denaturing PAGE of a protein over-expressed from the pEP21b-YFP-ZF (1) according to an exemplary embodiment. A and B indicate storage solutions in which proteins are dissolved;

FIG. 5 is a result of non-denaturing PAGE of a protein over-expressed from the pEP21b-YFP-ZF (2) according to an exemplary embodiment. A and B indicate storage solutions in which proteins are dissolved;

FIG. 6 is a result of gel mobility shift assay with ZF01 and a DNA fragment including AATTAG; and

FIG. 7 is a result of gel mobility shift assay with ZF02 and a DNA fragment including AACTGA.

### MODE FOR THE INVENTION

Reference will now be made in detail to embodiments, examples of which are illustrated in the accompanying drawings, wherein like reference numerals refer to the like elements throughout. In this regard, the present embodiments may have different forms and should not be construed as being limited to the descriptions set forth herein. Accordingly, the embodiments are merely described below, by referring to the figures, to explain aspects of the present description.

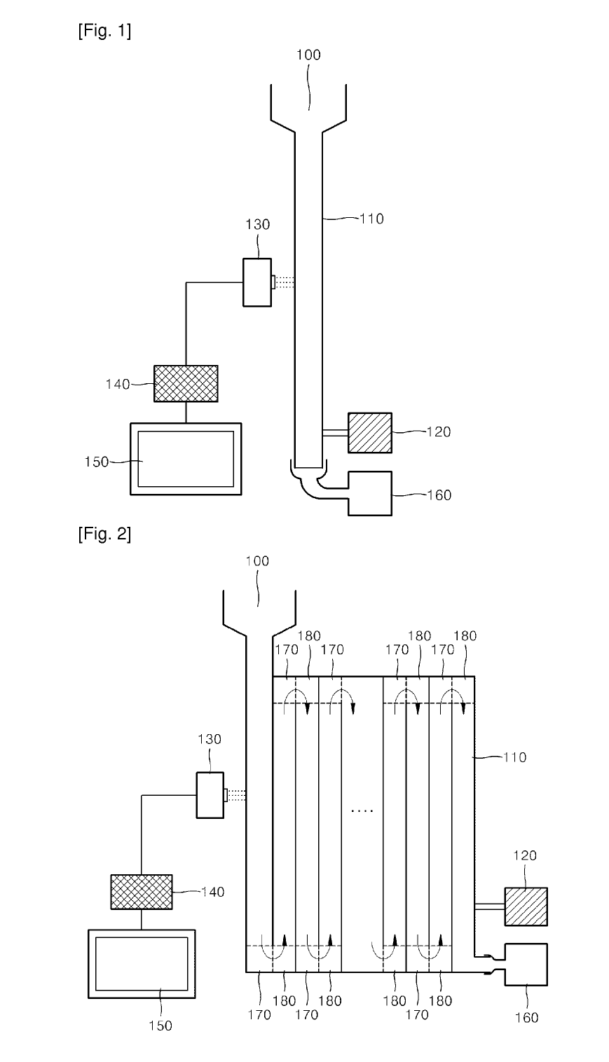

FIGS. 1 and 2 are schematic views of devices for determining a nucleotide sequence according to one exemplary embodiment. Referring to FIGS. 1 and 2, examples of a method for determining a nucleotide sequence by using a device for determining a nucleotide sequence according to one exemplary embodiment will be described as follows.

First, a sample including a target nucleic acid whose nucleotide sequence to be analyzed is injected into a sample injection unit 100. The sample may be, for example, a target nucleic acid including a buffer solution. In addition, the contacting of the sequence specific binding protein and the detectable tag and the target nucleic acid may be in advance performed in the sample injection unit 100.

The target nucleic acid injected into the sample injection unit 100 is transported into a sample transportation unit 110. The sample transportation unit 110 may be composed of one or two more channels. In addition, in order for one molecule of the target nucleic acid not to form a secondary structure in the channel, the channel may be manufactured to have a width and a depth (about 30 nm to about 200 nm) sufficient for the target nucleic acid to which the sequence specific binding protein and the detactable tag bind to be passed.

The target nucleic acid to be transported through the sample transportation unit 110 may be subjected to control of the transportation speed by a fluid flow control unit 120. In addition, the target nucleic acid may be disposed in the channel, in a state where the secondary structure has not been yet formed, by the fluid flow control unit 120. In this way, a detecting unit 130 may detect various detectable tags on the sequence specific binding protein binding to the nucleic acid when the target nucleic acid has been transported or in a stationary state where the target nucleic acid is disposed for the secondary structure not to be formed. For example, the detecting unit 130 may be a detection device which may detect fluorescence in various wavelengths by controlling the wavelengths.

A signal detected from the detecting unit 130 is subjected to converting the signal generated from the target nucleic acid into a nucleotide sequence which corresponds to the signal and a position of the signal in an operation unit 140. Subsequently, the converted nucleotide sequence may be identified by a user in an output unit 150. A target nucleic acid sample after being subjected to the determining of the nucleotide sequence may be finally transported into a sample waste unit 160 to be discarded.

As shown in FIG. 2, a device for determining a nucleotide sequence according to one exemplary embodiment may include at least two channels in a sample transportation unit 110. When at least two channels are included in the sample transportation unit 110, a sample recycling unit 170 and a sample labelling unit 180 may be further included in each channel. The target nucleic acid injected through the sample injection unit 100 as described above passes an initial channel of the sample transportation unit 100, leading to the sample recycling unit 170. An enzyme which may cleave proteins, for example, proteinase-K may be included in the sample recycling unit 170 to cleave a sequence specific binding protein and a detectable tag which bind to the target nucleic acid. At the time, a fluid flow control unit 120 may control the flow of a buffer solution including the target nucleic acid for the target nucleic acid to stay in the sample recycling unit 170. The target nucleic acid from which the sequence specific binding protein and the detectable tag has been completely removed is transported from the sample recycling unit 170 to the sample labelling unit 180 by a flow of the buffer solution. The sequence specific binding protein and the detectable tag bound in the previous step and a sequence specific binding protein and the detectable tag capable of recognizing another sequence may be included in the sample labelling unit 180. In addition, the sequence specific binding protein has a detectable tag connected to the protein and different from the tag in the previous step. Therefore, a sequence specific binding protein different from the protein in the previous step may bind to the target nucleic acid in the sample labelling unit 180 to determine a nucleotide sequence, in the next step, different from the sequence in the previous step. The direction of an arrow in FIG. 2 indicates a flow direction of the fluid.

The device may be manufactured to include a membrane which includes pores smaller than proteins (for example, proteinase K or the sequence specific binding protein) included in the sample recycling unit 170 and the sample labelling unit 180, and larger than the target nucleic acid, between the sample recycling unit 170 and the sample labelling unit 180 for only the target nucleic acid to be transported.

When the device for determining a nucleotide sequence, including at least two channels according to one exemplary embodiment, is subjected to the steps several times, the nucleotide sequence of a long target nucleic acid (for example, about 1 kb to about 10 Mb) may be determined at one time. In addition, when two or more sample transportation units 110 according to one exemplary embodiment are mounted, the nucleotide sequences of various types of nucleic acids may be determined at one time.

### EXAMPLE 1

### Preparation of Target Nucleic Acid for Determining Nucleotide Sequence

As a target nucleic acid for determining a nucleotide sequence, a human genome DNA was selected. The extraction of the human genome DNA was performed by using cells isolated from human blood, human mucosal epithelial cells, or cultured cells. A commercially available kit (Bio-rad) was used from the obtained cells to extract a human genome DNA according to protocols provided by the manufacturer. The kit may be used to extract a human genome DNA of about 250 kb or more on average.

### EXAMPLE 2

### Preparation of Sequence Specific Binding Protein Linked with Detectable Tag

Processes of constructing a vector to express a sequence specific binding protein linked with a detectable tag (YFP) and purifying the sequence specific binding protein by using the vector were described below.

In order to express the sequence specific binding protein, polynucleotide fragments coding for a (Gly2Ser)2 linker and a fluorescent protein (YFP) were obtained by polymerase chain reaction (PCR). The amplification of the polynucleotide fragments was performed using a template pEYFP (Invitrogen, USA), a YFP-BamHI-F primer (SEQ ID NO. 1) including a nucleotide sequence coding for the (Gly2Ser)2 linker and a nucleotide sequence that is cleavable by BamHI, and a YFP-XhoI-R primer (SEQ ID NO. 2) including a nucleotide sequence that is cleavable by XhoI. The amplification was performed using a GeneAmp PCR System 9700 (Applied Biosystem) under the following PCR conditions: at 95° C. for 5 minutes; repeated 30 times at 95° C. for 20 seconds and at 68° C. for 2 minutes; at 68° C. for 5 minutes; and cooled to 4° C. The resulting PCR product was washed using a QIAquick Multiwell PCR Purification kit (Qiagen) according to a manufacturer's protocol and was used in subsequent steps. The amplified PCR product was cleaved with BamHI and XhoI restriction enzymes and inserted into a pET21b (Novagen) vector, which was cleaved with the same restriction enzymes, to construct a pET21b-YFP vector.

A sequence specific binding protein was prepared to include two zinc finger motifs, and two sequence specific binding proteins including two zinc finger motifs may be designed by methods disclosed in http://www.scripps.edu/mb/barbas/zfdesign/zfdesignhome.php. The two sequence specific binding proteins were those which target AATTAG and AACTGA, respectively. The amino acid sequence in a zinc finger motif specifically recognizing AAT in the specific sequence AATTAG is TTGNLTV (SEQ ID NO. 3), and the amino acid sequence specifically recognizing TAG is REDNLHT (SEQ ID NO. 4). In addition, the amino acid sequence in a zinc finger motif specifically recognizing AAC in the specific sequence AACTGA is DSGNLRV (SEQ ID NO. 5), and the amino acid sequence specifically recognizing TGA is QAGHLAS (SEQ ID NO. 6). The amino acid sequences of the two sequence specific binding proteins were SEQ ID NOS. 7 and 8. Polynucleotide fragments coding for the sequence specific binding proteins were prepared by synthesizing oligonucleotides of sense and antisense strands corresponding to the polynucleotide fragments according to a method known to the art, followed by annealing. A nucleotide sequence that is cleavable by BamHI restriction enzyme at the 5′ and 3′ ends was added to each polynucleotide fragment, and inserted into the vector pET21b-YFP constructed as above. The oligonucleotides of the sense and antisense strands of the synthesized polynucleotide fragments were SEQ ID NO. 9 to SEQ ID NO. 12, respectively.

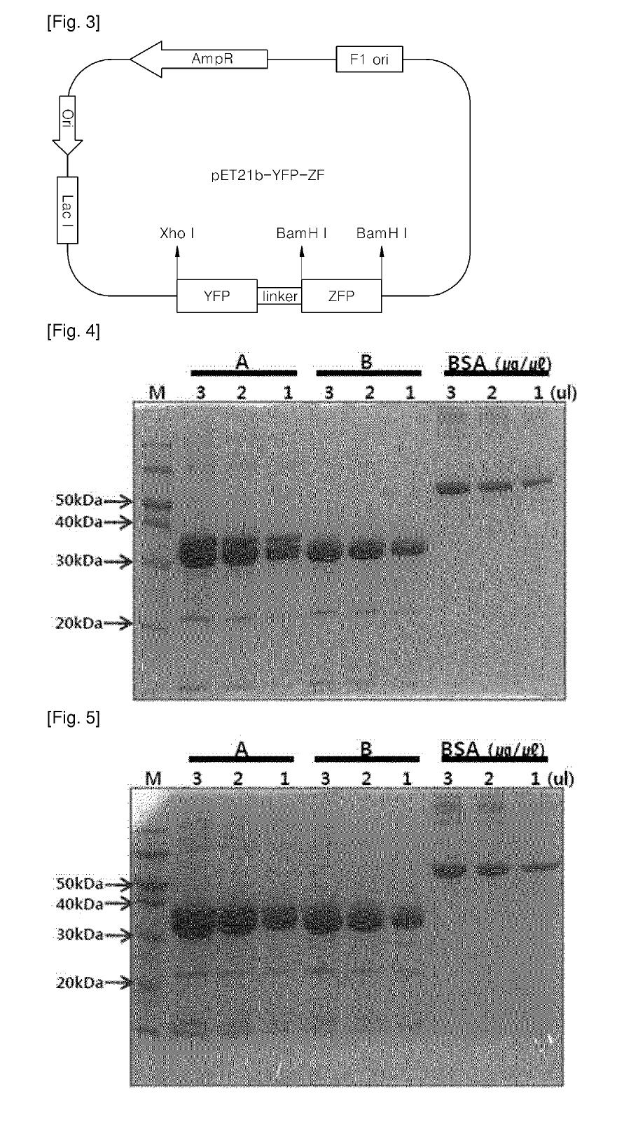

The synthesized polynucleotide fragments were cleaved with BamHI restriction enzyme and inserted into the vector pET21b-YFP constructed as above, which was cleaved with the same restriction enzyme, to prepare pET21b-YFP-ZF (1) and pET21b-YFP-ZF (2) (FIG. 3).

In order to use the prepared vector to over-express the protein, the vector was transformed in E. coli BL21 (DE3). A Luria Broth (LB) liquid medium to which 50 ug/ml of ampicillin was added was used as a culture medium. A 0.5 mM isopropyl-β-d-thiogalactopyranoside (IPTG) was added to the culture medium when the optical density (O.D., absorbance) reached a value of about 0.5 at a 600-nm wavelength, and the transformed E. coli BL21 (DE3) was further cultured at about 25° C. for about 16 hours. After being sonicated in a 25 mM Tris-HCl buffer solution (pH 8.0), the cultured cell was centrifuged (at 10,000×g) to obtain a supernatant. The supernatant was loaded on a Ni2+-NTA superflow column (Qiagen) equilibrated with the buffer solution, and was then washed with a wash buffer solution in a volume five times higher than that of the column. Then, an elution buffer solution (including 25 mM Tris-HCl (pH 8.0); 2.5 mM υ-mercaptoethanol; 125 mM imidazole; and 150 mM NaCl) was loaded to elute the protein. Fractions including the protein were collected and filtered using Amicon Ultra-15 Centrifugal Filters (Milipore) to remove salts therefrom. Then, the salt-removed fractions were concentrated. The concentrated protein (YFP-linker-ZFP fusion protein) was dissolved and stored in a storage solution A (including 25 mM Tris-HCl (pH 8.0); 2.5 mM β-mercaptoethanol; 125 mM imidazole; 150 mM NaCl; and 50% glycerol) or a storage solution B (including 20 mM Tris-HCl (pH 7.5); 1 mM DTT; 100 mM NaCl; and 50% glycerol). The concentration of the purified protein was quantified using bovine serum albumin (BSA) as a standard material. FIGS. 4 and 5 showed that the fusion protein has a molecular weight of about 30 kDa and was separated with a high purity. Hereinafter, a protein expressed from the pET21b-YFP-ZF (1) is referred to as ZF01, and a protein expressed from the pET21-YFP-ZF (2) is referred to as ZF02.

### EXAMPLE 3

### Identification of Binding Capability of Target Nucleic Acid and Sequence Specific Binding Protein

In order to identify whether the sequence specific binding protein prepared in Example 2 specifically binds to the target nucleic acid, a gel mobility shift assay was performed.

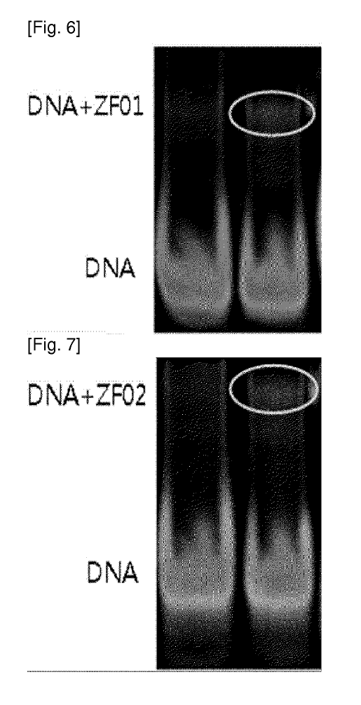

First, a 20-mer oligonucleotide including the nucleotide sequences AATTAG or AACTGA, which the sequence specific binding protein may specifically recognize, was synthesized (SEQ ID NO. 13 to SEQ ID NO. 16). Subsequently, each of the oligonucleotides of SEQ ID NOS. 13 and 14, SEQ ID NOS. 15 and 16 was annealed to prepare a target nucleic acid. The prepared target nucleic acid and the protein prepared in Example 2 was added to a buffer solution (including 10 ml of 20 mM bis-Tris propane (pH 7.0); 100 mM NaCl; 5 mM MgCl₂; 20 mM ZnSO₄; 10% glycerol; 0.1% Nonidet P-40; 5 mM DTT; and 0.10 mg/ml BSA), followed by reaction at room temperature for about 1 hour. The reactant was subjected to non-denaturing PAGE and as a result as shown in FIG. 6 and FIG. 7, it was identified that a combined product of a target nucleic acid 1 (a polynucleotide fragment including AATTAG) or 2 (a polynucleotide fragment including AACTGA) with ZF01 or ZF02 respectively recognizing the nucleic acid 1 or 2 was shorter in mobility distance on the gel than a negative control group (target nucleic acid).

### EXAMPLE 4

### Process of Determining Nucleotide Sequence of Target Nucleic Acid

0.4 pM of the target nucleic acid of about 250 kb obtained in Example 1 and 10 pM of the protein prepared in Example 2 (ZF01 or ZF02) was added to a buffer solution (including 10 ml of 20 mM bis-Tris propane (pH 7.0); 100 mM NaCl; 5 mM MgCl₂; 20 mM ZnSO₄; 10% glycerol; 0.1% Nonidet P-40; 5 mM DTT; and 0.10 mg/ml BSA), followed by reaction at room temperature for about 1 hour. Subsequently, YOYO-1 was added to the reactant, left for about 10 minutes to stain the backbone of the target nucleic acid, followed by injection into a nano-channel (a channel manufactured to have a width of about 100 nm, a depth of about 80 nm, and a length of about 1 mm in a quadrangular form into silicon subjected to a surface treatment with SiO₂) to detect a fluorescent signal from the YFP of the sequence specific binding protein using a spectrophotometer at about 491 nm to about 509 nm.

### EXAMPLE 5

### Reanalysis Test of Nucleotide Sequence

The target nucleic acid, analyzed in Example 4, was removed from the nano-channel to treat the nucleic acid with a DNase-free proteinase K. The target nucleic acid was reacted with the sequence specific binding protein ZF01 in the same manner as in Example 4, and a fluorescent signal was measured from the YFP of the sequence specific binding protein. As a result, it was identified that the sequence specific binding protein ZF01 bound to the target nucleic acid has been removed. Subsequently, the sequence specific binding protein ZF02 was again reacted in the same manner as above, and a fluorescent signal was again detected therefrom. As a result, it was confirmed that the ZF01 has been removed from the target nucleic acid and the sequence specific binding protein ZF02 has been bound thereto. This suggested that the recycling of a nucleic acid may be performed in a method for determining a nucleotide sequence according to one exemplary embodiment, indicating that the information of nucleotide sequence may be efficiently obtained from only a small amount of a target nucleic acid.

In accordance with a kit including a sequence specific binding protein according to one exemplary embodiment and a method and device for determining a nucleotide sequence of a target nucleic acid by using the kit, the nucleotide sequence of the target nucleic acid may be more efficiently determined.

It should be understood that the exemplary embodiments described therein should be considered in a descriptive sense only and not for purposes of limitation. Descriptions of features or aspects within each embodiment should typically be considered as available for other similar features or aspects in other embodiments.

### Sequence Listing Free Text

The sequences of the nucleotide or polypeptide of SEQ ID NO: 1 through SEQ ID NO: 16 are filed as the Sequence Listing, and contexts in the Sequence Listing are incorporated into this application in their entities.

1. A kit for determining a nucleotide sequence of a target nucleic acid comprising: at least one sequence specific binding protein and a detectable tag.

2. The kit of claim 1, wherein the target nucleic acid has a length of 1 kb to 10 Mb.

3. The kit of claim 1, wherein the target nucleic acid is double stranded.

4. The kit of claim 1, wherein the sequence specific binding protein comprises at least one motif selected from the group consisting of a zinc finger motif, a helix-turn-helix motif, a helix-loop-helix motif, a leucine zipper motif, a nucleic acid-binding motif of restriction endonuclease, and a combination thereof.

5. The kit of claim 1, wherein the detectable tag comprises at least one selected from the group consisting of a colored bead, a chromophore, a fluorescent material, a fluorescent protein, a phosphorescent material, an electrically detectable molecule, a molecule providing modified fluorescence-polarization or modified light-diffusion, a quantum dotand a combination thereof.

6. The kit of claim 5, wherein the fluorescent protein is selected from the group consisting of a yellow fluorescent protein (YFP), a green fluorescent protein (GFP), a red fluorescent protein (RFP) and a combination thereof.

7. The kit of claim 1, wherein the detectable tag is linked to the sequence specific binding protein by a linker.

8. A gene construct comprising a polynucleotide encoding a fluorescent protein fused with a zinc finger protein, operatively linked to a promoter.

9. The gene construct of claim 8, wherein the fluorescent protein is selected from the group consisting of a yellow fluorescent protein (YFP), a green fluorescent protein (GFP), a red fluorescent protein (RFP) and a combination thereof.

10. The gene construct of claim 8, wherein the fluorescent protein and the zinc finger protein are fused in order, N terminus to C terminus.

11. The gene construct of claim 8, wherein the fluorescent protein and the zinc finger protein are fused by peptide linker.

12. A device for determining a nucleotide sequence of a target nucleic acid, comprising: a sample injection unit for injecting a target nucleic acid and a sequence specific binding protein and a detectable tag; a sample transportation unit comprising a channel fluidically connected to the sample injection unit; a fluid flow control unit for controlling a flow of the sample; and a detecting unit for detecting a signal from the detectable tag.

13. The device of claim 12, wherein the device further comprises a sample waste unit fluidically connected to the sample transportation unit and disposed in the opposite end of a channel to which the sample injection unit is connected.

14. The device of claim 12, wherein the sample transportation unit allows one end of each channel in at least two channels to be sequentially and fluidically connected to the other end.

15. The device of claim 12, wherein the device further comprises a sample recycling unit fluidically connected to one end of the channel.

16. The device of claim 15, wherein the sample recycling unit further-comprise a proteolytic enzyme.

17. The device of claim 12, wherein the device further comprises a sample labelling unit fluidically connected to the sample recycling unit, disposed at the other end of the channel to which the sample recycling unit is connected.

18. The device of claim 17, wherein the sample labelling unit further comprise sequence specific binding protein and a detectable tag.

19. The device of claim 12, wherein the channel has a width and a depth of about 30 to about 200 nm.

20. The device of claim 12, wherein the device further comprises an operation unit for converting a signal detected from the detectable tag into a nucleotide sequence which corresponds to the signal.

21. The device of claim 12, wherein the sequence specific binding protein comprises at least one motif which is selected from the group consisting of zinc finger motif, a helix-turn-helix motif, a helix-loop-helix motif, a leucine zipper motif, a nucleic acid-binding motif of restriction endonuclease, and a combination thereof.

22. The device of claim 12, wherein the sequence specific binding protein comprises two zinc finger motifs.

23. The device of claim 12, wherein the detectable tag comprises at least one selected from the group consisting of a colored bead, a chromophore, a fluorescent material, a fluorescent protein, a phosphorescent material, an electrically detectable molecule, a molecule providing modified fluorescence-polarization or modified light-diffusion, and a quantum dot and a combination thereof.

24. The device of claim 23, wherein the fluorescent protein is selected from the group consisting of a yellow fluorescent protein (YFP), a green fluorescent protein (GFP), a red fluorescent protein (RFP) and a combination thereof.

25. The method of claim 23, wherein the channel is a nano- to micro-channel.

26. The method of claim 25, wherein the sequence specific binding protein comprises at least one motif which is selected from the group consisting of zinc finger motif, a helix-turn-helix motif, a helix-loop-helix motif, a leucine zipper motif, a nucleic acid-binding motif of restriction endonuclease, and a combination thereof.

27. The method of claim 25, wherein the nucleic acid is double-stranded.

28. The method of claim 25, wherein the nucleic acid has a length of about 1 kb to about 10 Mb.

29. The method of claim 25, wherein the sequence specific binding protein comprises two zinc finger motifs.

30. The method of claim 25, wherein the detecting comprises detecting the position where the motif binds to the nucleic acid.

31. The method of claim 25, wherein the determining comprises combining information about the position where the motif binds to the nucleic acid and the sequence to which the motif binds.

32. The method of claim 25, the detectable tag comprises at least one selected from the group consisting of a colored bead, a chromophore, a fluorescent material, a fluorescent protein, a phosphorescent material, an electrically detectable molecule, a molecule providing modified fluorescence-polarization or modified light-diffusion, and a quantum dot and a combination thereof.

33. The method of claim 32, wherein the fluorescent protein is selected from the group consisting of a yellow fluorescent protein (YFP), a green fluorescent protein (GFP), a red fluorescent protein (RFP) and a combination thereof.


SEQUENCE LISTING (16 sequences)

SEQ ID NO: 1 - DNA, 27 bp, Artificial Sequence
  YFP-BamHI-F primer
ggatccatggtgagcaagggcgaggag

SEQ ID NO: 2 - DNA, 27 bp, Artificial Sequence
  YFP-XhoI-R primer
ctcgagttacttgtacagctcgtccat

SEQ ID NO: 3 - protein, 7 aa, Artificial Sequence
  amino acid sequence which specifically recognize “AAT”
TTGNLTV

SEQ ID NO: 4 - protein, 7 aa, Artificial Sequence
  amino acid sequence which specifically recognize “TAG”
REDNLHT

SEQ ID NO: 5 - protein, 7 aa, Artificial Sequence
  amino acid sequence which specifically recognize “AAC”
DSGNLRV

SEQ ID NO: 6 - protein, 7 aa, Artificial Sequence
  amino acid sequence which specifically recognize “TGA”
QAGHLAS

SEQ ID NO: 7 - protein, 64 aa, Artificial Sequence
  amino acid sequence of zinc finger motif which specifically recognize “AATTAG”
LEPGEKPYKCPECGKSFSREDNLHTHQRTHTGEKPYKCPECGKSFSTTGNLTVHQRTHTGKKTS

SEQ ID NO: 8 - protein, 64 aa, Artificial Sequence
  amino acid sequence of zinc finger motif which specifically recognize “AACTGA”
LEPGEKPYKCPECGKSFSQAGHLASHQRTHTGEKPYKCPECGKSFSDSGNLRVHQRTHTGKKTS

SEQ ID NO: 9 - DNA, 192 bp, Artificial Sequence
  DNA sequence(sense) of zinc finger motif which specifically recognize “AATTAG”
ctggaaccgggtgaaaaaccgtacaaatgcccggaatgcggtaaatctttctctcgtgaagacaacctgcacacccaccagcgtacccacaccggtgaaaaaccgtacaaatgcccggaatgcggtaaatctttctctaccaccggtaacctgaccgttcaccagcgtacccacaccggtaaaaaaacctct

SEQ ID NO: 10 - DNA, 192 bp, Artificial Sequence
  DNA sequence(antisense) of zinc finger motif which specifically recognize “AATTAG”
agaggtttttttaccggtgtgggtacgctggtgaacggtcaggttaccggtggtagagaaagatttaccgcattccgggcatttgtacggtttttcaccggtgtgggtacgctggtgggtgtgcaggttgtcttcacgagagaaagatttaccgcattccgggcatttgtacggtttttcacccggttccag

SEQ ID NO: 11 - DNA, 192 bp, Artificial Sequence
  DNA sequence(sense) of zinc finger motif which specifically recognize “AACTGA”
ctggaaccgggtgaaaaaccgtacaaatgcccggaatgcggtaaatctttctctcaggcgggtcacctggcgtctcaccagcgtacccacaccggtgaaaaaccgtacaaatgcccggaatgcggtaaatctttctctgactctggtaacctgcgtgttcaccagcgtacccacaccggtaaaaaaacctct

SEQ ID NO: 12 - DNA, 192 bp, Artificial Sequence
  DNA sequence(antisense) of zinc finger motif which specifically recognize “AACTGA”
agaggtttttttaccggtgtgggtacgctggtgaacacgcaggttaccagagtcagagaaagatttaccgcattccgggcatttgtacggtttttcaccggtgtgggtacgctggtgagacgccaggtgacccgcctgagagaaagatttaccgcattccgggcatttgtacggtttttcacccggttccag

SEQ ID NO: 13 - DNA, 20 bp, Artificial Sequence
  oligonucleotide for EMSA including “AATTAG” (sense strand)
tttttttaattagttttttt

SEQ ID NO: 14 - DNA, 20 bp, Artificial Sequence
  oligonucleotide for EMSA including “AATTAG” (antisense strand)
aaaaaaattaatcaaaaaaa

SEQ ID NO: 15 - DNA, 20 bp, Artificial Sequence
  oligonucleotide for EMSA including “AACTGA” (sense strand)
tttttttaactgattttttt

SEQ ID NO: 16 - DNA, 20 bp, Artificial Sequence
  oligonucleotide for EMSA including “AACTGA” (antisense strand)
aaaaaaattgactaaaaaaa